(19) United States
(12) Patent Application Publication
(10) Pub. No.: US 2016/0220095 A1
(43) Pub. Date: Aug. 4, 2016

(54) PROCESSOR DEVICE FOR ENDOSCOPE, ENDOSCOPE SYSTEM, AND CONTACTLESS POWER SUPPLY METHOD FOR ENDOSCOPE SYSTEM

(71) Applicant: FUJIFILM Corporation, Tokyo (JP)

(72) Inventor: Koji SHIMOMURA, Kanagawa (JP)

(73) Assignee: FUJIFILM Corporation, Tokyo (JP)

(21) Appl. No.: 14/981,960

(22) Filed: Dec. 29, 2015

(30) Foreign Application Priority Data
Feb. 3, 2015 (JP) 2015-019000

(51) Int. Cl. A61B 1/00; A61B 1/07; A61B 1/045; A61B 1/06

(52) U.S. Cl. CPC A61B 1/00006; A61B 1/00013; A61B 1/00029; A61B 1/00045; A61B 1/00009; A61B 1/0661; A61B 1/07; A61B 1/045

(57) ABSTRACT

A power supply control unit starts power supply to an endoscope if an external trigger is applied (Step S10) (Step S12). If the power supply to the endoscope is started, the endoscope becomes operable. As a result, voltage information showing a current voltage value V_{rect} is transmitted from the endoscope to the processor device for an endoscope through optical communication as feedback information. A control unit of the processor device for an endoscope receives the voltage information showing the current voltage value V_{rect}, and outputs the received voltage information to a power supply control unit (Step S14). The power supply control unit adjusts the frequency of an electric current made to flow to a power transmitting coil of a power supply unit, on the basis of the received voltage information (Steps S16 to S24).

### CROSS-REFERENCE TO RELATED APPLICATIONS

The present application claims priority under 35 U.S.C. §119 to Japanese Patent Application No. 2015-019000, filed on Feb. 3, 2015. The above application(s) is hereby expressly incorporated by reference, in its entirety, into the present application.

### BACKGROUND OF THE INVENTION

1. Field of the Invention

The present invention relates to a processor device for an endoscope, an endoscope system, and a contactless power supply method for an endoscope system, and particularly, to the technique of supplying power in a contactless manner to an endoscope.

2. Description of the Related Art

Endoscope systems are constituted of an imaging unit, such as a charge coupled device (CCD) image sensor, which images the inside of a body cavity, an endoscope including a connector section provided at an end of the universal cord, a connector section on which the connector section of the endoscope is detachably mounted, a control unit that performs image processing or the like on image signals output from the endoscope, and a processor device for an endoscope including a light source.

In the endoscope systems, supply of power from the processor device for an endoscope to the endoscope, and transmission of image signals and control signals between the processor device for an endoscope and the endoscope are performed by connecting the connector section of the endoscope, and the connector section of the processor device for an endoscope by electrical contacts.

In the endoscope systems, it is necessary to perform cleaning or disinfection of the endoscope after use. Therefore, it is necessary to attach a waterproofing cap that protects the electrical contacts to the connector section of the endoscope. However, not only is substantial time and effort required for attachment and detachment of the waterproofing cap, but also there is a problem that the electrical contacts may be damaged when attachment of the waterproofing cap is forgotten.

In order to cope with such problems, an endoscope system described in JP2013-208187A makes the connector section of the endoscope a waterproof structure, provides a communication unit that performs short-distance radio communication of image signals between the endoscope and the processor device for an endoscope, and provides a power supply unit that supplies power in a contactless manner from the processor device for an endoscope to the endoscope.

Additionally, the processor device for an endoscope described in JP2013-208187A is provided with a connector support part that detachably supports the connector section of the endoscope, a locking mechanism that performs switching between a locked state in which a support state of the connector section by the connector support part is held and an unlocked state where holding is released, and a short-distance contactless power transmission control unit that enables power supply of power in a contactless manner from a power supply unit when the locking mechanism is in the locked state and disables supply of power when the locking mechanism is in the unlocked state. Accordingly, the processor device for an endoscope can supply power in a contactless manner only when the connector section of the endoscope is mounted on the connector support part of the processor device for an endoscope.

### SUMMARY OF THE INVENTION

Meanwhile, the processor device for an endoscope is unable to immediately ascertain the power situation of the endoscope when power is supplied in a contactless manner from the processor device for an endoscope to the endoscope, and there is a problem in that power becomes insufficient or excessive due to the load of the endoscope when constant power is supplied.

The invention has been made in view of such circumstances, and an object thereof is to provide a processor device for an endoscope, an endoscope system, and a contactless power supply method for an endoscope system that can excellently perform contactless supply of power to the endoscope.

In order to achieve the above object, a processor device for an endoscope according to an aspect of the invention includes a power supply unit including a power transmitting coil that supplies power to an endoscope in a contactless manner; a first optical communication unit that performs optical communication with the endoscope; an external trigger receiving unit that receives an external trigger showing a start instruction for power supply to the endoscope; and a power supply control unit that controls the power supply unit. The power supply control unit starts power supply from the power supply unit to the endoscope in the case where the external trigger receiving unit receives the external trigger. The first optical communication unit starts optical communication with a second optical communication unit provided in the endoscope and receives power reception information corresponding to an excess or deficiency in the amount of received power of the endoscope through optical communication from the second optical communication unit, in the case where the power supply is started. The power supply control unit controls the power supply unit on the basis of the power reception information received by the first optical communication unit, and adjusts the power to be transmitted from the power supply unit.

According to the aspect of the invention, if the external trigger is received and the power supply to the endoscope is started, optical communication with the endoscope is allowed. As a result, the power reception information corresponding to an excess or deficiency in the amount of received power of the endoscope is received through the optical communication from the endoscope. The power supply control unit controls the power supply unit on the basis of the received power reception information, and adjusts the power to be transmitted from the power supply unit. Accordingly, supply of the power required by the endoscope to the endoscope can be excellently performed through contactless power supply irrespective of the load fluctuation of the endoscope.

In the processor device for an endoscope according to another aspect of the invention, it is preferable that the external trigger receiving unit includes a mounting detection unit that detects whether or not the endoscope has been mounted on the processor device for an endoscope, and receives the time when the mounting of the endoscope has been detected as the external trigger in the case where the mounting detection unit detects the mounting of the endoscope. Accordingly, in the case where the endoscope is mounted on the processor device for an endoscope, contactless supply of power can be automatically performed.

In the processor device for an endoscope according to still another aspect of the invention, it is preferable that the external trigger receiving unit includes a test start switch that makes an instruction for the start of endoscopy, and receives a manual operation time of the test start switch as the external trigger if the test start switch is manually operated. Accordingly, if the test start switch is manually operated according to a user's intention, contactless supply of power can be performed.

In the processor device for an endoscope according to still another aspect of the invention, it is preferable that the power supply unit supplies power to the endoscope in a contactless manner using an electromagnetic resonance method, and the power supply control unit controls the frequency of an electric current made to flow to the power transmitting coil of the power supply unit, on the basis of the power reception information received by the first optical communication unit, and adjusts the power to be transmitted from the power supply unit.

In the processor device for an endoscope according to still another aspect of the invention, it is preferable that the power reception information is voltage information showing a direct current voltage corresponding to the load state of the endoscope, and the power supply control unit controls the frequency of the electric current made to flow to the power transmitting coil such that the electric current moves in a direction approaching a resonant frequency at which the power supply unit and the power receiving unit of the endoscope magnetically resonate with each other, in the case where the voltage information received by the first optical communication unit is lower than a first threshold value, and controls the frequency of the electric current made to flow to the power transmitting coil such that electric current moves in a direction away from the resonant frequency, in the case where the voltage information received by the first optical communication unit is higher than a second threshold value.

In the processor device for an endoscope according to still another aspect of the invention, it is preferable that the first threshold value is a value that is smaller than the second threshold value by a hysteresis width. Accordingly, the searching when the frequency of the electric current made to flow to the power transmitting coil is adjusted can be prevented.

In the processor device for an endoscope according to still another aspect of the invention, it is preferable to further include an image signal receiving unit that receives an image signal, which has been captured by the endoscope, in a contactless manner from an image signal transmitting unit provided in the endoscope; an image processing unit that performs image processing on the image signal received by the image signal receiving unit; and an output unit that outputs the image signal, which has been subjected to image processing by the image processing unit, to a display. Accordingly, the image signal can be received in a contactless manner from the endoscope, and the connector section of the endoscope can be made to have a waterproof structure.

In the processor device for an endoscope according to another aspect of the invention, it is preferable to further include a light source that supplies light for illumination to a light guide of the endoscope via a mounting unit on which the endoscope is mounted.

An endoscope system according to still another aspect of the invention includes the above processor device for an endoscope; and an endoscope connected to the processor device for an endoscope. The endoscope includes a power receiving unit including a power receiving coil that performs power reception in a contactless manner from the processor device for an endoscope; and a second optical communication unit that starts optical communication with the first optical communication unit provided in the processor device for an endoscope and transmits the power reception information corresponding to an excess or deficiency in the amount of received power of the endoscope through optical communication, in the case where power is supplied via the power receiving unit.

In the endoscope system according to still another aspect of the invention, it is preferable that the processor device for an endoscope includes a control unit that controls an imaging operation of the endoscope, and the control unit transmits a control signal for controlling the imaging operation of the endoscope from the first optical communication unit to the second optical communication unit.

In the endoscope system according to still another aspect of the invention, it is preferable that the endoscope includes an image signal transmitting unit that transmits an image signal, which has been captured by the endoscope, in a contactless manner to the image signal receiving unit provided in the processor device for an endoscope.

A contactless power supply method for an endoscope system according to still another aspect of the invention includes, in an endoscope system including a processor device for an endoscope having a power supply unit including a power transmitting coil, and a first optical communication unit, and an endoscope having a power receiving unit including a power receiving coil, and a second optical communication unit, a step of starting power supply from the power supply unit to the power receiving unit of the endoscope in the case where an external trigger receiving unit of the processor device for an endoscope receives an external trigger showing a start instruction for power supply to the endoscope; a step of causing, in the case where power supply is performed via the power receiving unit, the second optical communication unit to start optical communication with the first optical communication unit provided in the processor device for an endoscope and receive power reception information corresponding to an excess or deficiency in the amount of received power of the endoscope through optical communication; and a step of causing a power supply control unit provided in the processor device for an endoscope to control the power supply unit on the basis of the power reception information received via the first optical communication unit, and adjust the power to be transmitted from the power supply unit.

In the contactless power supply method for an endoscope system according to still another aspect of the invention, it is preferable that the professor device for an endoscope supplies power to the endoscope in a contactless manner using an electromagnetic resonance method, and in the case where the external trigger receiving unit receives the external trigger, the power supply control unit starts power supply from the power supply unit to the power receiving unit of the endoscope, acquires the power reception information from the second optical communication unit via the first optical communication, controls the frequency of an electric current made to flow to the power transmitting coil of the power supply unit on the basis of the acquired power reception information, and adjusts the power to be transmitted from the power supply unit.

According to the invention, the power reception information corresponding to an excess or deficiency in the amount of received power of the endoscope is received through optical communication with the endoscope, and the power to be transmitted in a contactless manner from the power supply unit to the endoscope using the received power reception information as feedback information is adjusted. Therefore, irrespective of the load fluctuation of the endoscope, supply of the power required by the endoscope to the endoscope can be excellently performed by contactless power supply.

### BRIEF DESCRIPTION OF THE DRAWINGS

FIG. 1 is an appearance view illustrating an endoscope system.

FIG. 2 is a block diagram illustrating the internal configuration of the endoscope system.

FIG. 3 is an appearance view of a connector section of an endoscope.

FIG. 4 is a view illustrating an embodiment of a mounting detection unit that detects mounting of the connector section of the endoscope.

FIG. 5 is a configuration view illustrating a power receiving unit of the endoscope and a power supply unit of a processor device for an endoscope, which are illustrated in FIG. 1.

FIG. 6 is a flowchart illustrating a contactless power supply method for the endoscope system.

FIG. 7A and FIG. 7B are a graph illustrating the relationship between the frequencies of electric currents made to flow to a power transmitting coil and direct current voltages corresponding to the load states of the endoscope, with respect to time.

FIG. 8 is a side view of main parts of the processor device for an endoscope used in order to explain another embodiment of the mounting detection unit that detects the mounting of the connector section of the endoscope.

### DESCRIPTION OF THE PREFERRED EMBODIMENTS

Hereinafter, embodiments of a processor device for an endoscope, an endoscope system, and a contactless power supply method for an endoscope system according to the invention will be described with reference to the accompanying drawings.

[Endoscope System]

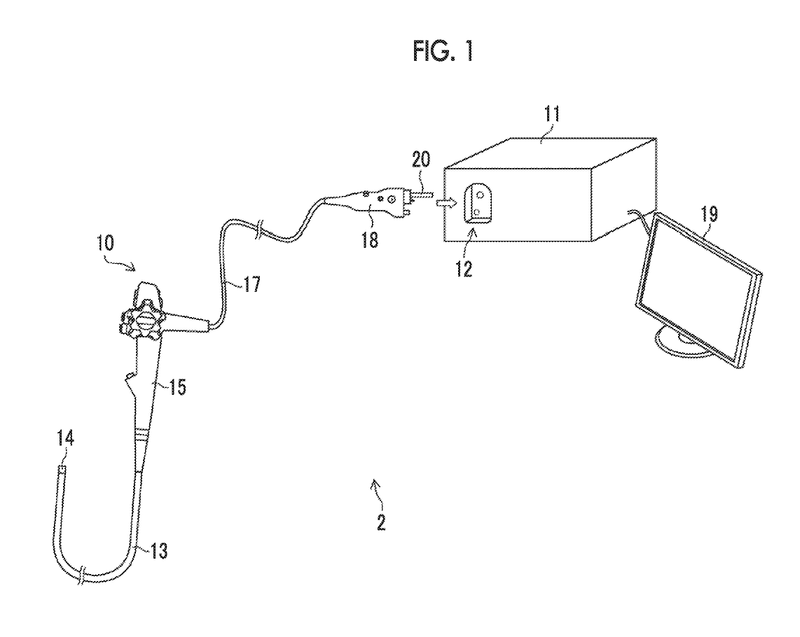

FIG. 1 is an appearance view of the endoscope system according to the invention.

As illustrated in FIG. 1, the endoscope system 2 is constituted of an endoscope 10 and a processor device 11 for an endoscope.

The endoscope 10 is illustrated as a flexible mirror, and includes a flexible insertion section 13 inserted into a patient's body cavity, an operation section 15 disposed at a base end of the insertion section 13, a universal cord 17 disposed at the operation section 15, and a connector section 18 provided at an end of the universal cord 17 and connected to a connector section 12 that functions as a mounting unit of the processor device 11 for an endoscope. However, the endoscope 10 is not limited to the flexible mirror, and the invention can be applied to any other types of endoscopes, such as a hard mirror.

An observation window, an illumination window, and the like are provided at a tip surface of the insertion section 13. An objective optical system that focuses photographic subject light from a part to be observed taken in by the observation window as an optical image, an imaging unit that converts the optical image focused by the objective optical system into electrical signals, and the like are arranged in a tip part 14 that constitutes the tip of the insertion section 13.

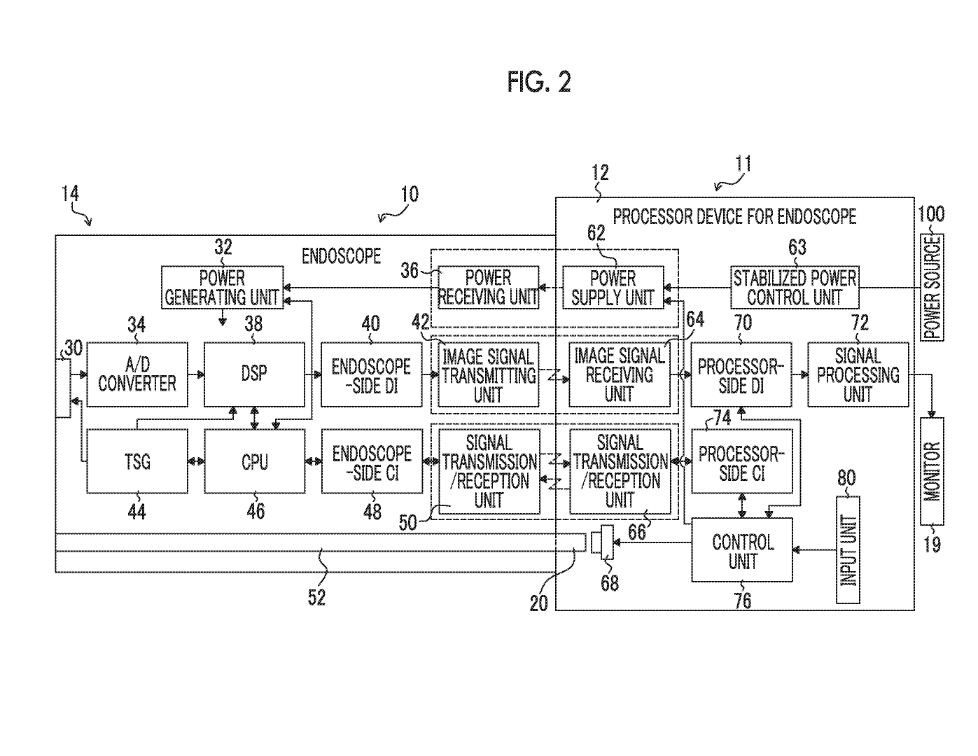

Image signals output from the imaging unit are transmitted to an image signal transmitting unit 42 (FIG. 2) by a transmission cable arranged to be inserted through the endoscope to the connector section 18 via the insides of the insertion section 13, the operation section 15, and the universal cord 17. The image signals are converted into light signals by the image signal transmitting unit 42, and are optically transmitted to the processor device 11 for an endoscope in a contactless manner.

Additionally, a light emitting part of a light guide 52 (FIG. 2) that transmits light for irradiating a part to be observed from the illumination window is arranged at the tip part 14. The light guide 52 is arranged to be inserted through the endoscope to the connector section 18 via the insides of the insertion section 13, the operation section 15, and the universal cord 17. Additionally, a light guide rod 20 coupled to the light guide 52 protrudes from the connector section 18.

A release button for recording an endoscope image as a still image, and the like, other than an angle knob for adjusting the orientation of the tip surface of the insertion section 13 in vertical and horizontal directions and a gas and water supply button for jetting air or water from the tip surface of the insertion section 13, are provided in the operation section 15. The orientation of the tip surface of the insertion section 13 is adjusted by bending a bending part provided in the vicinity of a base end side of the tip part 14.

The universal cord 17 is covered with an outer wall part that is tubular, elongated, and flexible, and the transmission cable arranged to be inserted through cavity parts inside the insertion section 13 and the operation section 15, the light guide 52, a gas and water supply tube, and the like are arranged in a lumen inside the outer wall part so as to be inserted therethrough.

FIG. 2 is a block diagram illustrating the internal configuration of the endoscope system 2 of FIG. 1.

The connector section 18 of the endoscope 10 is connected to the connector section 12 of the processor device 11 for an endoscope. Power reception and power supply of power, transmission and reception of image signals, and transmission and reception of various kinds of control signal are performed in a contactless manner between the connector section 18 of the endoscope 10 and the connector section 12 of the processor device 11 for an endoscope.

Therefore, the connector section 18 of the endoscope 10 is provided with a power receiving unit 36 that receives power in a contactless manner, an image signal transmitting unit 42 that optically transmits image signals of an imaging unit 30 in a contactless manner, and a signal transmission/reception unit 50 that optically transmits and receives control signals that control the imaging unit 30, and power reception information used for control of contactless power supply in a contactless manner and that functions as a second optical communication unit.

As described above, the connector section 18 of the endoscope 10 is connected to the connector section 12 of the processor device 11 for an endoscope. The processor device 11 for an endoscope performs supply (power supply) of power to the endoscope 10 connected (mounted) to the connector section 12, and transmission and reception of various signals to and from the endoscope 10.

The processor device 11 for an endoscope includes a light source 68. Light for illumination from the light source 68 is supplied to the light guide 52 via the light guide rod 20, and the light is transmitted from the light guide 52 to the tip part 14.

The connector section 12 of the processor device 11 for an endoscope connected to the connector section 18 of the endoscope 10 is provided with a power supply unit 62 that supplies power to the power receiving unit 36 of the endoscope 10 in a contactless manner, an image signal receiving unit 64 that receives the image signals from the image signal transmitting unit 42 of the endoscope 10 in a contactless manner, and a signal transmission/reception unit 66 that transmits and receives signals in a contactless manner to and from the signal transmission/reception unit 50 of the endoscope 10 and that functions as a first optical communication unit.

Additionally, the processor device 11 for an endoscope takes in the image signals output from the imaging unit 30 of the tip part 14 of the endoscope 10, performs various kinds of signal processing on the taken-in image signals, and generates image data that constructs a video (dynamic image) or a still image of a part to be observed. Then, the generated image data is output to a display (monitor) 19 connected by a cable, and the image or the like of the part to be observed is displayed on the monitor 19. Additionally, the generated image data is recorded on a recording medium if necessary.

The endoscope 10 is detachably mounted (connected) to the connector section 12 of the processor device 11 for an endoscope by the connector section 18. In the endoscope system 2 of the present embodiment, by virtue of the mounting between the connector section 18 of the endoscope 10 and the connector section 12 of the processor device 11 for an endoscope, an internal circuit of the endoscope 10 and an internal circuit of the processor device 11 for an endoscope are connected together by a contactless device, such as a transformer or a photo-coupler, via these connector sections. Accordingly, the electrical insulation between the internal circuit of the endoscope 10 and the internal circuit of the processor device 11 for an endoscope is ensured. That is, the invention is configured so that optical communication of control signals, contactless power supply of power, and optical communication of image signals can be realized.

Power required for driving of the internal circuit of the endoscope 10 is supplied from the processor device 11 for an endoscope by contactless power supply means consisting of the power supply unit 62 in the processor device 11 for an endoscope and the power receiving unit 36 in the endoscope 10. The power receiving unit 36 is arranged at the connector section 18 of the endoscope 10, and the power supply unit 62 is arranged at the connector section 12 of the processor device 11 for an endoscope.

The contactless power supply means is means for transmitting and receiving power in a contactless manner using electromagnetic coupling. If the connector section 18 of the endoscope 10 is mounted on the connector section 12 of the processor device 11 for an endoscope, the power supply unit 62 and the power receiving unit 36 are arranged close to each other at a distance allowing electromagnetic coupling, and are brought into a state where contactless power supply from the power supply unit 62 to the power receiving unit 36 is allowed. A commercial power source 100 is connected to the power supply unit 62 via the stabilized power control unit 63 outside the processor device 11 for an endoscope. The power supplied from the commercial power source 100 and stabilized by the stabilized power control unit 63 is supplied to the power supply unit 62. By the power supplied from the stabilized power control unit 63 to the power supply unit 62, the power is supplied from the power supply unit 62 to the power receiving unit 36 in a contactless manner. The power receiving unit 36 receives the power in a contactless manner from the power supply unit 62. In addition, the details of this contactless supply of power will be described below.

The endoscope 10 includes a power generating unit 32 connected to the power receiving unit 36, and the power generating unit 32 generates various kinds of driving power required by an internal circuit including the imaging unit 30 and the like, and supplies the generated power. For example, the power generating unit 32 has an electric current induced in the power receiving unit 36 inputs thereto, and generates the driving power to be supplied to an internal circuit including the imaging unit 30, a central processing unit (CPU) 46, and the like from the input electric current.

The imaging unit 30 is arranged at the tip part 14 of the endoscope 10. The imaging unit 30 is a device that converts the light of the optical image of the part to be observed, which has been taken in by the observation window and focused by the objective optical system, into electrical signals, and outputs the converted electrical signals as image signals, as described above. As the imaging unit 30, for example, a solid-state imaging element, such as a charge coupled device (CCD) image sensor or a complementary metal oxide semiconductor (CMOS) image sensor, can be included.

In the present embodiment, transmission and reception of image signals between the endoscope 10 and the processor device 11 for an endoscope are performed by contactless optical communication means. The image signals output from the imaging unit 30 are transmitted by contactless optical transmission via the connector section 12 of the processor device 11 for an endoscope from the connector section 18 of the endoscope 10. In the present embodiment, an analog/digital converter (A/D converter) 34, a digital signal processor (DSP) 38, and a timing signal generator (TSG) 44, and the like are provided in order to process the image signals from the imaging unit 30. The image signals from the imaging unit 30 are changed from analog signals to digital signals by the A/D converter 34. Image signals output from the A/D converter 34 are transmitted to the DSP 38. The DSP 38 performs required processing, such as amplification, gamma correction, white balance processing, and the like on the image signals from the A/D converter 34.

For example, the following configuration is provided in order to perform contactless optical transmission between the endoscope 10 and the processor device 11 for an endoscope. The endoscope 10 includes an endoscope-side digital interface (DI) 40 connected to the DSP 38, and the image signal transmitting unit 42 connected to endoscope-side DI 40. The image signals processed by the DSP 38 is transmitted to the image signal transmitting unit 42 via the endoscope-side DI 40. Processing is performed on the image signals from the imaging unit 30, and light signals are transmitted from the image signal transmitting unit 42 to the processor device 11 for an endoscope according to the processed image signals. The image signal transmitting unit 42 may be a light emitting device that can radiate light for optical communication, and can include a laser light emitting element, a light emitting diode, or the like. The laser light emitting element means an element that radiates laser light that is coherent light, and includes a gas laser, a solid-state laser, a semiconductor laser, or the like.

The processor device 11 for an endoscope includes the image signal receiving unit 64 that receives the light signals from the image signal transmitting unit 42, a processor-side DI 70 connected to the image signal receiving unit 64, and a signal processing circuit 72 that functions as an image processing unit and an output unit that are connected to the processor-side DI 70. The image signal receiving unit 64 is a light receiving device that converts the received light signals into electrical signals, and can include, for example, a light-receiving element, such as a photodiode or a semiconductor device such as a phototransistor. The image signals, which have been optically received by the image signal receiving unit 64 and converted into electrical signals, are converted into image signals for display by the signal processing circuit 72 via the processor-side DI 70, and are output to the monitor 19. In addition, signal processing, such as gamma correction and white balance processing, may be performed by the signal processing circuit 72 of the processor device 11 for an endoscope, without being limited to a case where the signal processing is performed by the DSP 38 of the endoscope 10.

In the present embodiment, image signal transmission/reception means based on contactless optical communication is constituted of the image signal transmitting unit 42 and the image signal receiving unit 64. In the image signal transmitting unit 42 that transmits the image signals of the imaging unit 30 in a contactless manner, and the image signal receiving unit 64 that receives signals from the image signal transmitting unit 42 in a contactless manner, a wireless communication method can be used, without being limited to the contactless optical communication (optical wireless communication method). The optical wireless communication method means a method that transmits and receives signals using infrared rays or the like and the wireless communication method means a method that transmits and receives signals using wireless communication (electromagnetic waves).

If the connector section 18 of the endoscope 10 is mounted on the connector section 12 of the processor device 11 for an endoscope, the image signal transmitting unit 42 and the image signal receiving unit 64 are arranged close to each other at a distance allowing optical communication, and are set to a state where contactless optical communication from the image signal transmitting unit 42 to the image signal receiving unit 64 is allowed.

Transmission and reception of control signals between the endoscope 10 and the processor device 11 for an endoscope are performed through contactless optical communication. The TSG 44 and the CPU 46 are connected to the imaging unit 30. The TSG 44 and the CPU 46 output a driving signal for allowing the imaging unit 30 to acquire the image signals to the imaging unit 30. An endoscope-side communication interface (CI) 48 and the signal transmission/reception unit 50 are connected to the CPU 46. The signal transmission/reception unit 50 is a device that performs contactless optical transmission and reception of the control signals between the endoscope 10 and the processor device 11 for an endoscope, and includes a light emitting device that optically transmits the control signals to the processor device 11 for an endoscope as light signals, and a light receiving device that receives the control signals from the processor device 11 for an endoscope as light signals. The signal transmission/reception unit 50 can include, for example, contactless optical data communication based on Infrared Data Association (IrDA) provided with an infrared light emitting element that optically transmits signals (infrared rays), and a light receiving element (a photodiode, a phototransistor, or the like) that optically receives signals. At least the signal transmission/reception unit 50 is arranged at the connector section 18 of the endoscope 10. Other devices, for example, the endoscope-side CI 48 and the like, may be arranged at the connector section 18 of the endoscope 10.

The processor device 11 for an endoscope includes the signal transmission/reception unit 66 that optically transmits and receives the control signals between the signal transmission/reception unit 50 of the endoscope 10 and a processor-side CI 74 connected to the signal transmission/reception unit 66. The signal transmission/reception unit 66 is a device that can optically transmit and receive the control signals between the endoscope 10 and the processor device 11 for an endoscope, and includes a light emitting device that optically transmits the control signals to the endoscope 10 as light signals, and a light receiving device that receives the control signals from the endoscope 10 as light signals. The signal transmission/reception unit 66 of the processor device 11 for an endoscope can include, for example, contactless optical data communication based on IrDA provided with an infrared light emitting element that optically transmits signals different from those of the signal transmission/reception unit 50 of the endoscope 10 (infrared rays), and a light receiving element (a photodiode, a phototransistor, or the like) that optically receives signals different from those of the signal transmission/reception unit 50. Generally, infrared rays mean electromagnetic waves that have a wavelength of 0.7 μm to 1 mm.

If the connector section 18 of the endoscope 10 is mounted on the connector section 12 of the processor device 11 for an endoscope, the signal transmission/reception units 50 and 66 are arranged close to each other at a distance allowing optical communication, and are set to a state where contactless optical transmission and reception are allowed between the signal transmission/reception units 50 and 66.

The processor device 11 for an endoscope includes the light source 68. The light source 68 can include, for example, a xenon lamp, or a semiconductor device, such as a laser diode or a light emitting diode. The endoscope 10 includes the light guide 52. The light guide rod 20 connected to the light guide 52 is provided at an end of the light guide 52. The light guide rod 20 protrudes from the connector section 18, and is connected to the connector section 12 of the processor device 11 for an endoscope. If the connector section 18 of the endoscope 10 is mounted on the connector section 12 of the processor device 11 for an endoscope, the light guide rod 20 and the light source 68 are aligned with each other, and the light from the light source 68 is transmitted to the tip part 14 via the light guide rod 20 and the light guide 52.

Additionally, the processor device 11 for an endoscope includes a control unit 76, and an input unit 80 including an operation switch, a test start switch, a keyboard, a mouse, and the like, and the control unit 76 controls the entire endoscope system 2 generally according to operator's the operations input from the input unit 80. In addition, the test start switch included in the input unit 80 and the control unit 76 function as an external trigger receiving unit.

For example, the control unit 76 controls the power supply unit 62, the light source 68, the processor-side DI 70, and the like, sends control signals for controlling imaging operation and the like to the CPU 46 and the like that constitute the internal circuit of the endoscope 10, and controls the entire endoscope system 2.

Moreover, the control unit 76 transmits control signals or the like based on a user's instruction input in which the user indicates power ON or OFF of the processor device 11 for an endoscope by the input unit 80, to the CPU 46 of the endoscope 10 through the signal transmission/reception units 66 and 50.

Additionally, the control signals from the CPU 46 of the endoscope 10 are transmitted to the control unit 76 of the processor device 11 for an endoscope through signal transmission/reception units 66 and 50 and the processor-side CI 74, and the control unit 76 controls the processor device 11 for an endoscope according to the control signals.

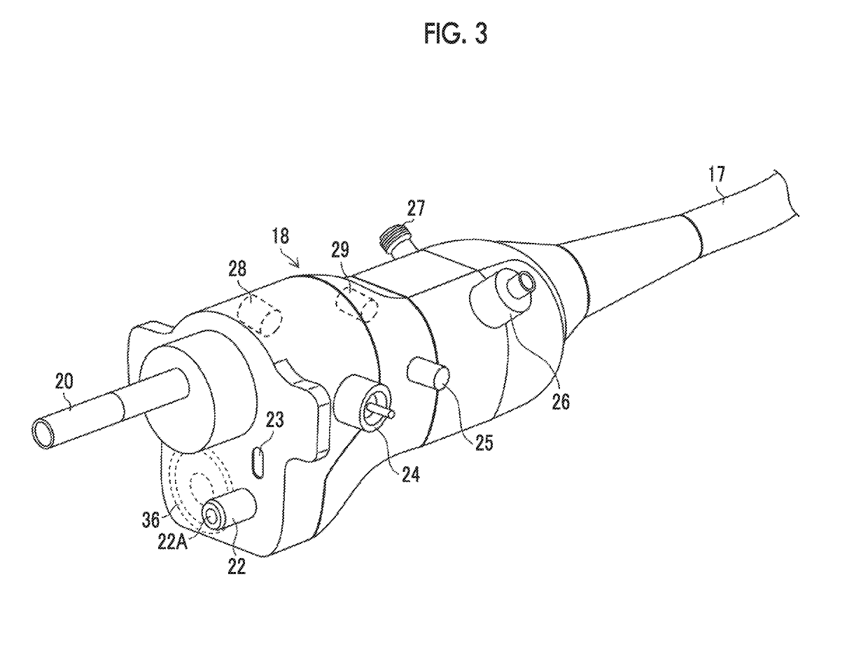

FIG. 3 is an appearance view illustrating the connector section 18 of the endoscope 10. As described above, the endoscope 10 and the processor device 11 for an endoscope perform power reception and power supply of power, transmission and reception of image signals, and transmission and reception of control signals in both directions, in a contactless manner. It is not necessary to provide the connector section 18 with an electrical contact directly connected to the processor device 11 for an endoscope.

Therefore, it is possible to provide a waterproof structure in which the connector section 18 of the endoscope 10 is covered with resin that has electrical insulation and has excellent chemical resistance. By making the connector section 18 into a waterproof structure, electrical components or the like inside the connector section 18 can be protected from cleaning water or the like, it is not necessary to attach a separate waterproofing cap in the case of cleaning or disinfection, and it is suitable particularly when the endoscope 10 is cleaned and sterilized by a high-pressure steam sterilizer (autoclave device).

As illustrated in FIG. 3, the connector section 18 of the endoscope 10 includes the light guide rod 20 that protrudes from the connector section 18, and a shaft 22.

The connector section 18 has a tubular shape, and the above-described power receiving unit 36, image signal transmitting unit 42, and signal transmission/reception unit 50 are arranged in a space inside the connector section.

The shaft 22 is used for the alignment between the image signal transmitting unit 42 of the endoscope 10 and the image signal receiving unit 64 of the processor device 11 for an endoscope. Particularly, the image signal transmitting unit 42 is arranged in an extending direction of a central axis of the shaft 22. A window 22A is provided at the tip of the shaft 22 in order to allow light to be transmitted therethrough. Image signals are optically transmitted and received in a contactless manner between the image signal transmitting unit 42 and the image signal receiving unit 64 via the window 22A.

Additionally, a window 23 is provided at a position corresponding to the signal transmission/reception unit 50 in a connecting surface of the connector section 18. Control signals are optically transmitted and received in a contactless manner between the signal transmission/reception units 50 and 66 via the window 23.

The power receiving unit 36 is arranged at a position near the connecting surface of the connector section 18. The power receiving unit 36 is arranged inside the connector section 18, and is not exposed to the outside.

Additionally, a gas and water supply connector 24, a balloon connector 25, a ventilation connector 26, a connector 27 used when an electrosurgical unit (electric scalpel) or the like is used, a suction connector 28, and a sub-water supply connector 29 are arranged on a side surface of the connector section 18.

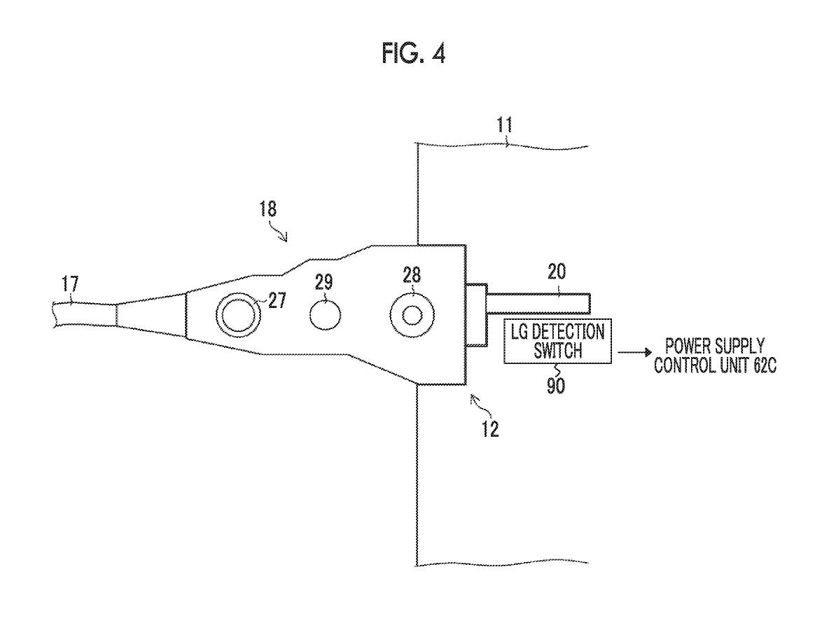

FIG. 4 is a view illustrating an embodiment of a mounting detection unit that detects mounting of the connector section 18 of the endoscope 10, and is a side view of main parts of the connector section of the processor device 11 for an endoscope.

As illustrated in FIG. 4, a light guide (LG) detection switch 90 is disposed in the vicinity of the connector section of the processor device 11 for an endoscope on which the connector section 18 of the endoscope 10 is mounted.

The LG detection switch 90 detects electrical connection with the light guide rod 20 covered with metal, thereby detecting that the light guide rod 20 has been inserted (that is, that the connector section 18 of the endoscope 10 has been mounted on the processor device 11 for an endoscope). If insertion of the light guide rod 20 is detected, the LG detection switch 90 outputs the detection signal to a power supply control unit 62C or a control unit 76 (to be described below) as an external trigger.

In addition, as the LG detection switch 90, a microswitch that detects mechanical contact with the light guide rod 20 without being limited to the electrical connection with the light guide rod 20, a photointerrupter that detects the presence/absence of the light guide rod 20 optically, or the like may be used.

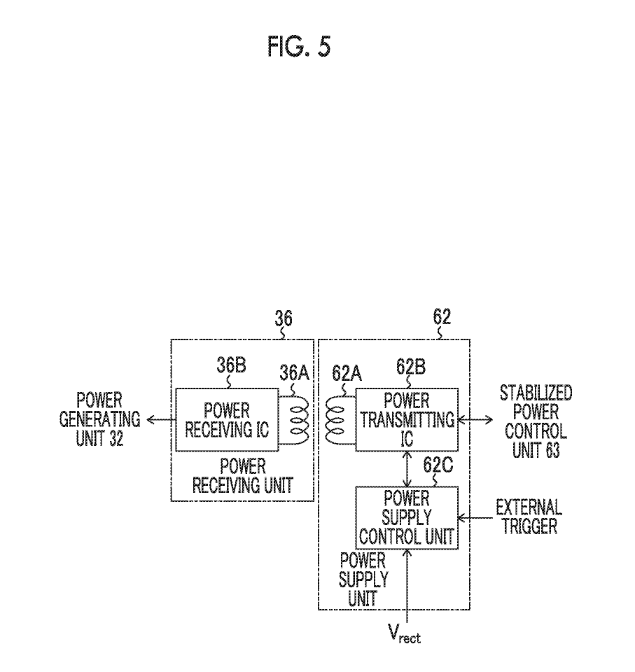

FIG. 5 is a configuration view illustrating the power receiving unit 36 of the endoscope 10 and the power supply unit 62 of the processor device 11 for an endoscope that are illustrated in FIG. 1.

The power supply unit 62 is constituted of a power transmitting coil 62A, a power transmitting integrated circuit (IC) 62B, and a power supply control unit 62C.

The stabilized power from the stabilized power control unit 63 is supplied to the power transmitting IC 62B, and the power transmitting IC 62B supplies a variable frequency current (high-frequency current) to the power transmitting coil 62A by using the power input from the stabilized power control unit 63 as power.

The power supply control unit 62C controls the power transmitting IC 62B that constitutes the power supply unit 62 and that adjusts the power transmitted from the power supply unit 62. If a detection signal (external trigger) showing mounting of the endoscope 10 is applied from the LG detection switch 90, the test start switch that makes an instruction for the start of endoscopy of the input unit 80 is manually operated, and if an instruction input (external trigger) is applied via the control unit 76, the power supply unit 62 is actuated to start power supply to the endoscope 10.

Additionally, the power supply control unit 62C controls the power transmitting IC 62B on the basis of power reception information that is applied via the control unit 76 and corresponds to an excess or deficiency in the amount of received power of the endoscope 10, and adjusts the power transmitted from the power supply unit 62. In the present embodiment, a voltage value (voltage information) V_{rect} showing a direct current voltage rectified after power has been received by the power receiving unit 36 of the endoscope 10 and a direct current voltage fluctuating in response to the load state of the endoscope 10 are used as the above power reception information, and the power supply control unit 62C controls the frequency of an electric current made to flow from the power transmitting IC 62B to the power transmitting coil 62A such that the voltage value V_{rect} falls within a desired voltage range.

The power receiving unit 36 of the endoscope 10 is constituted of a power receiving coil 36A and a power receiving IC 36B. The power receiving coil 36A supplies power to the endoscope 10 in a contactless manner using an electromagnetic resonance method, and is magnetically coupled with a power transmitting coil 62A, and an induced current resulting from an alternating field (magnetic flux) caused by a high-frequency current that flows into the power transmitting coil 62A is generated in the power receiving coil 36A.

The power receiving IC 36B includes a rectifier circuit, and rectifies the induced current caused in the power receiving coil 36A using the rectifier circuit and then outputs the rectified current to the power generating unit 32.

The power generating unit 32 illustrated in FIG. 2 generates various kinds of driving power required by the internal circuit of the endoscope 10, using the power received by the power receiving unit 36 as mentioned above as power, and supplies the generated power.

The CPU 46 acquires the voltage value V_{rect} for a reference power in the power generating unit 32 as voltage information from the power generating unit 32, and notifies the processor device 11 for an endoscope of the acquired voltage information as feedback information via the endoscope-side CI 48 and the signal transmission/reception unit 50 that performs contactless optical communication.

In addition, the voltage value V_{rect} fluctuates in response to the load state of the endoscope 10, and fluctuates depending on the power transmitted from the power supply unit 62.

[Contactless Power Supply Method for Endoscope System]

Next, the contactless power supply method for the endoscope system 2 having the above configuration will be described.

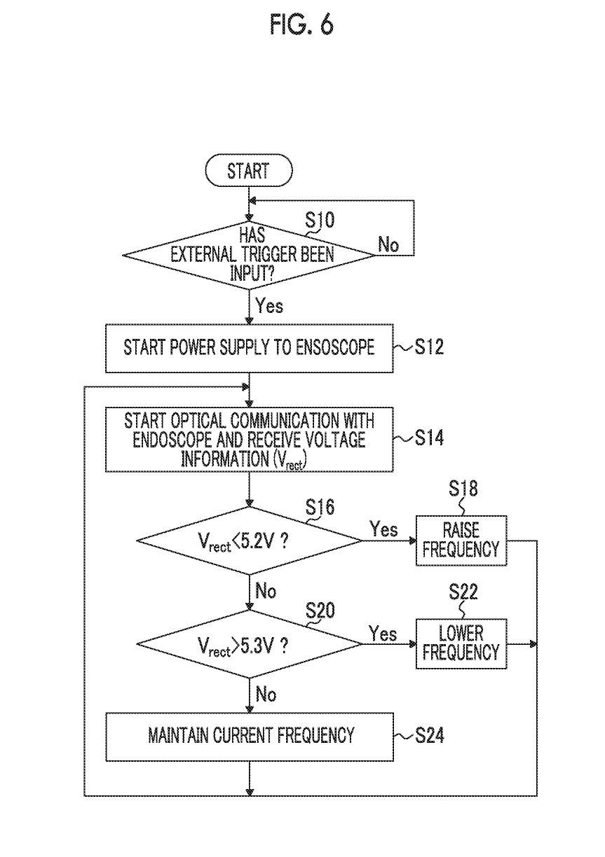

FIG. 6 is a flowchart illustrating the contactless power supply method for the endoscope system, and illustrating mainly the operation of the processor device 11 for an endoscope.

In FIG. 6, if the power supply control unit 62C determines whether or not an external trigger showing a start instruction for power supply to the endoscope 10 has been input (Step S10), and the external trigger has been input (when the determination result is “Yes”), the power supply to the endoscope 10 is started (Step S12).

Here, the external trigger is applied from the LG detection switch 90 to the power supply control unit 62C when the LG detection switch 90 has detected insertion of the light guide rod 20 (namely, when mounting of the endoscope 10 has been detected).

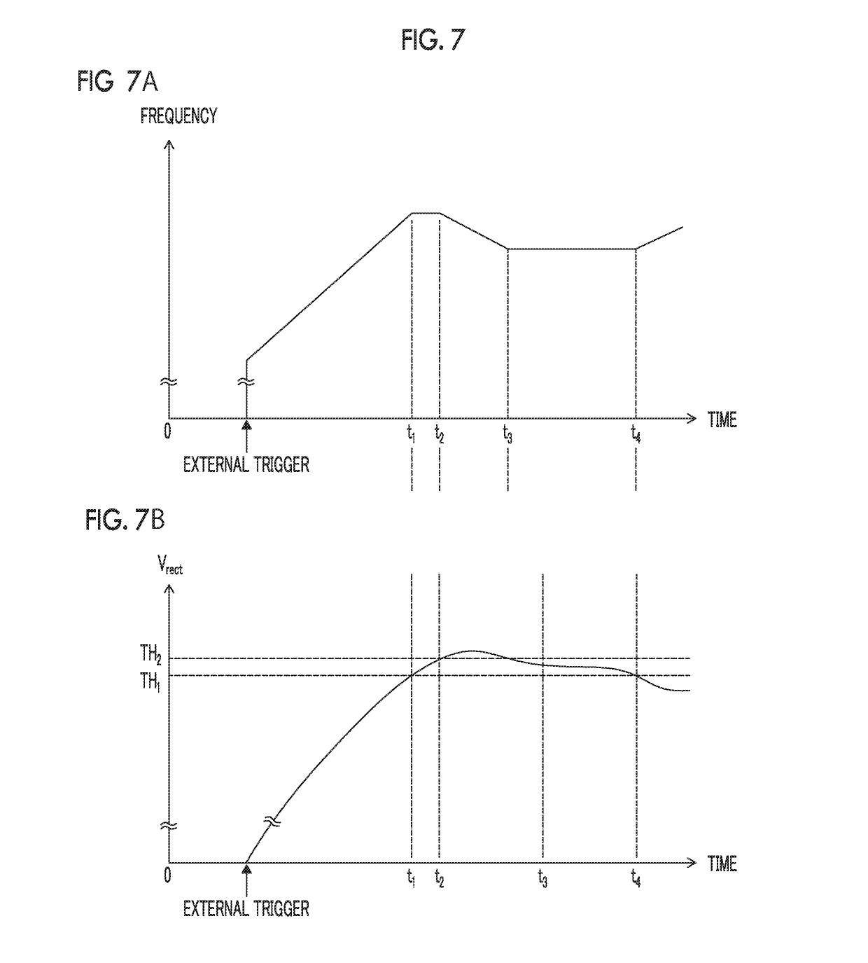

Additionally, the power supply when the power supply to the endoscope 10 is started, as illustrated in FIG. 7A, is performed by raising (sweeping) the frequency of an electric current made to flow to the power transmitting coil 62A of the power supply unit 62 slowly from a frequency sufficiently lower than a resonant frequency at which the power supply unit 62 and the power receiving unit 36 magnetically resonate with each other from the time when the external trigger has been input. Accordingly, the power supplied from the power supply unit 62 to the power receiving unit 36 becomes slowly larger. As a result, as illustrated in FIG. 7B, the voltage value V_{rect} showing the direct current voltage caused in the endoscope 10 also becomes larger in accordance with the supplied power.

If the power supply to the endoscope 10 is started, the endoscope 10 is automatically powered on, or is powered on by a control signal applied through optical communication from the processor device 11 for an endoscope, and supply of power to the internal circuit of the processor device 11 for an endoscope is started.

If the endoscope 10 is powered on, the CPU 46, the signal transmission/reception unit 50, and the like become operable. As a result, the endoscope 10 and the processor device 11 for an endoscope start bidirectional optical communication via the signal transmission/reception units 50 and 66 (Step S14). Then, voltage information showing a current voltage value V_{rect} from the endoscope 10 is transmitted to the processor device 11 for an endoscope through optical communication as feedback information, and the control unit 76 of the processor device 11 for an endoscope receives voltage information showing the current voltage value V_{rect} (acquisition), and outputs the acquired voltage information to the power supply control unit 62C (Step S14).

The power supply control unit 62C determines whether or not the current voltage value V_{rect} is smaller than a first threshold value TH₁ (5.2 V in the present embodiment) using the voltage information acquired via the control unit 76 (Step S16). In Step S16, if it is determined that the voltage value V_{rect} is smaller than 5.2 V (in the case of “Yes”), the power supply control unit 62C controls the frequency of the electric current made to flow to the power transmitting coil 62A of the power supply unit 62, and raises the frequency in a direction approaching the resonant frequency (Step S18). Accordingly, the power supplied to the endoscope 10 can be increased, and the voltage value V_{rect} of the power of the endoscope 10 can be raised.

Meanwhile, if the power supply control unit 62C determines that the current voltage value V_{rect} is equal to or more than 5.2 V (in the case of “No”), it is further determined whether or not the current voltage value V_{rect} is larger than a second threshold value TH₂ (5.3 V in the present embodiment) (Step S20). In Step S20, if it is determined that the voltage value V_{rect} is larger than 5.3 V (in the case of “Yes”), the power supply control unit 62C controls the frequency of the electric current made to flow to the power transmitting coil 62A of the power supply unit 62, and lowers the frequency in a direction away the resonant frequency (Step S22). Accordingly, the power supplied to the endoscope 10 can be decreased, and the voltage value V_{rect} of the power of the endoscope 10 can be lowered.

In Step S20, if it is determined that the voltage value V_{rect} is equal to or smaller than 5.3 V (in the case of “No”), the power supply control unit 62C maintains the frequency of the electric current made to flow to the power transmitting coil 62A of the power supply unit 62 (Step S24). Accordingly, the power supplied to the endoscope 10 is also maintained.

The processing of the above Step S14 to Step S24 is continuously performed at given time intervals.

Next, the control of power (the frequency of the electric current made to flow to the power transmitting coil 62A of the power supply unit 62) supplied in a contactless manner from the power supply unit 62 will be described referring to FIGS. 7A and 7B.

If an external trigger is input as illustrated in FIGS. 7A and 7B, the power supply control unit 62C slowly raises the frequency of the electric current made to flow to the power transmitting coil 62A from a frequency sufficiently lower than the resonant frequency. The voltage value V_{rect} rises with the rise of this frequency. At a time t₁, if the voltage value V_{rect} reaches the first threshold value TH₁, the frequency at the time t₁ is maintained.

Thereafter, at a time t₂, if the voltage value V_{rect} exceeds the second threshold value TH₂, the frequency is lowered slowly from the maintained frequency, and at a time t₃, if the voltage value V_{rect} reaches the second threshold value TH₂, the frequency at the time t₃ is maintained.

Subsequently, the load of the endoscope 10 becomes larger due to the start of imaging by the endoscope 10, or the like. As a result, at a time t₄, if the voltage value V_{rect} becomes smaller than the first threshold value TH₁ due to a voltage drop resulting from the load, the frequency is slowly raised from the maintained frequency.

In this way, by adjusting the frequency (supplied power) of the electric current made to flow to the power transmitting coil 62A of the power supply unit 62 corresponding to a change (particularly a change exceeding the range between the first threshold value TH₁ and the second threshold value TH₂) in the voltage value V_{rect} of the endoscope 10, an excess or deficiency in power can be prevented from occurring with respect to the load fluctuation of the endoscope 10.

In addition, the hysteresis width (0.1 V in the present embodiment) is set between the first threshold value TH₁ and the second threshold value TH₂, and this prevents searching when the frequency of the electric current made to flow to the power transmitting coil 62A is adjusted. Here, the hysteresis width is a dead band, and when the voltage value V_{rect} of the endoscope 10 falls within a range of the hysteresis width, switching of adjustment of the frequency of the electric current made to flow to the power transmitting coil 62A is not performed.

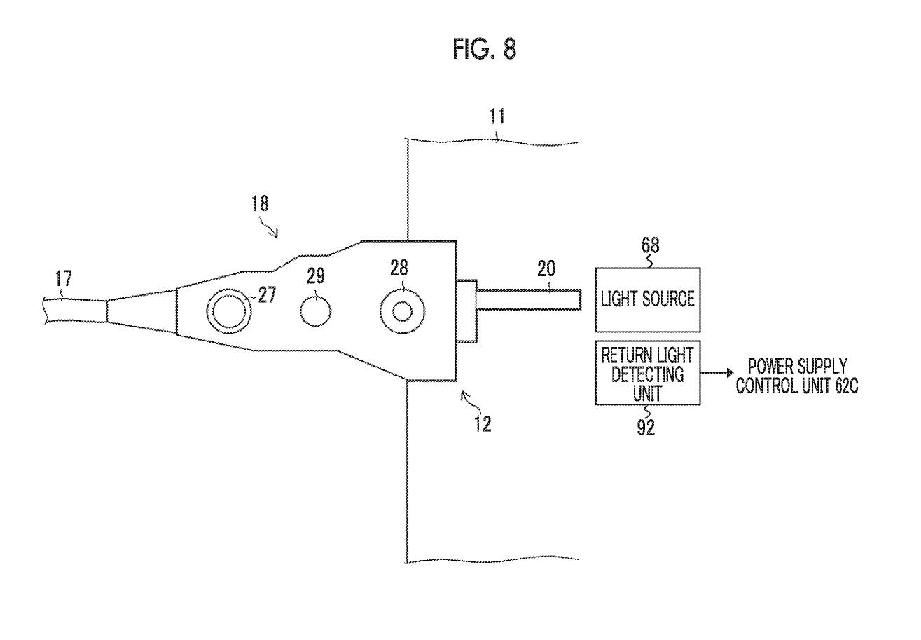

FIG. 8 is a view illustrating another embodiment of the mounting detection unit that detects the mounting of the connector section 18 of the endoscope 10, and a side view of main parts of the processor device 11 for an endoscope.

As illustrated in FIG. 8, a return light detecting unit 92 is disposed in the vicinity of the light source 68 of the processor device 11 for an endoscope on which the connector section 18 of the endoscope 10 is mounted.

The return light detecting unit 92 detects illumination light, which enters an end surface of the light guide rod 20 from the light source 68, as the return light from the tip of the light guide 52 or the reflected light from the end surface of the light guide rod 20. The return light detecting unit 92 detects the return light of the illumination light, which has entered the end surface of the light guide rod 20, thereby detecting that the light guide rod 20 has been inserted (that is, that the connector section 18 of the endoscope 10 has been mounted on the processor device 11 for an endoscope). In addition, when the light guide rod 20 is not inserted, the return light detecting unit 92 cannot receive return light, and accordingly can detect the presence/absence of insertion of the light guide rod 20.

If insertion of the light guide rod 20 is detected, the return light detecting unit 92 outputs the detection signal to the aforementioned power supply control unit 62C or control unit 76 as an external trigger.

[Others]

In the present embodiment, a case where contactless power supply is started by using the detection time of mounting of the endoscope detected by the mounting detection unit represented by the LG detection switch 90, the return light detecting unit 92, and the like as an external trigger, and a case where contactless power supply is started by using the manual operation time of the test start switch that makes an instruction for the start of the endoscopy of the input unit 80 as an external trigger have been described. However, the external trigger from the mounting detection unit and the external trigger from the test start switch may be separately used as follows.

When the processor device 11 for an endoscope is powered on in a state where the endoscope 10 is mounted on the processor device 11 for an endoscope, only the external trigger from the test start switch is validated. This is because contactless power supply is automatically started in a state where the mounting of the endoscope 10 is incomplete. On the other hand, when the endoscope 10 is mounted on the processor device 11 for an endoscope in a state where the processor device 11 for an endoscope is powered on, only the external trigger from the mounting detection unit is validated. This is because, wasted contactless power supply resulting from the operation of the test start switch in a state where the endoscope 10 is not mounted can be prevented, and contactless power supply can be started in conjunction with the mounting of the endoscope 10.

Additionally, as well as a case where supplied power is controlled by controlling the frequency of the electric current made to flow to the power transmitting coil of the power supply unit, the supplied power may be controlled by adjusting the duty ratio of switching on and off of an inverter connected to the power transmitting coil.

Moreover, in the present embodiment, the voltage information showing the direct current voltage (voltage value V_{rect}) in response to the load state of the endoscope is received from the endoscope as the feedback information when controlling supplied power. However, the invention is not limited to this. For example, the endoscope may determine an excess or deficiency in power, and may transmit the determination result to the processor device for an endoscope through optical communication from the endoscope.

Additionally, it is obvious that the invention is not limited to the above-described embodiment, and various modifications can be made without departing from the spirit of the invention.

What is claimed is:

1. A processor device for an endoscope comprising:
a power supply unit including a power transmitting coil that supplies power to an endoscope in a contactless manner;
a first optical communication unit that performs optical communication with the endoscope;
an external trigger receiving unit that receives an external trigger showing a start instruction for power supply to the endoscope; and
a power supply control unit that controls the power supply unit,
wherein the power supply control unit starts power supply from the power supply unit to the endoscope in the case where the external trigger receiving unit receives the external trigger,
wherein the first optical communication unit starts optical communication with a second optical communication unit provided in the endoscope and receives power reception information corresponding to an excess or deficiency in the amount of received power of the endoscope through optical communication from the second optical communication unit, in the case where the power supply is started, and
wherein the power supply control unit controls the power supply unit on the basis of the power reception information received by the first optical communication unit, and adjusts the power to be transmitted from the power supply unit.

2. The processor device for an endoscope according to claim 1,
wherein the external trigger receiving unit includes a mounting detection unit that detects whether or not the endoscope has been mounted on the processor device for an endoscope, and receives the time when the mounting of the endoscope has been detected as the external trigger in the case where the mounting detection unit detects the mounting of the endoscope.

3. The processor device for an endoscope according to claim 1,
wherein the external trigger receiving unit includes a test start switch that makes an instruction for the start of endoscopy, and receives a manual operation time of the test start switch as the external trigger in the case where the test start switch is manually operated.

4. The processor device for an endoscope according to claim 1,
wherein the power supply unit supplies power to the endoscope in a contactless manner using an electromagnetic resonance method, and
wherein the power supply control unit controls the frequency of an electric current made to flow to the power transmitting coil of the power supply unit, on the basis of the power reception information received by the first optical communication unit, and adjusts the power to be transmitted from the power supply unit.

5. The processor device for an endoscope according to claim 2,
wherein the power supply unit supplies power to the endoscope in a contactless manner using an electromagnetic resonance method, and
wherein the power supply control unit controls the frequency of an electric current made to flow to the power transmitting coil of the power supply unit, on the basis of the power reception information received by the first optical communication unit, and adjusts the power to be transmitted from the power supply unit.

6. The processor device for an endoscope according to claim 3,
wherein the power supply unit supplies power to the endoscope in a contactless manner using an electromagnetic resonance method, and
wherein the power supply control unit controls the frequency of an electric current made to flow to the power transmitting coil of the power supply unit, on the basis of the power reception information received by the first optical communication unit, and adjusts the power to be transmitted from the power supply unit.

7. The processor device for an endoscope according to claim 4,
wherein the power reception information is voltage information showing a direct current voltage corresponding to the load state of the endoscope, and
wherein the power supply control unit controls the frequency of the electric current made to flow to the power transmitting coil such that the electric current moves in a direction approaching a resonant frequency at which the power supply unit and the power receiving unit of the endoscope magnetically resonate with each other, in the case where the voltage information received by the first optical communication unit is lower than a first threshold value, and controls the frequency of the electric current made to flow to the power transmitting coil such that electric current moves in a direction away from the resonant frequency, in the case where the voltage information received by the first optical communication unit is higher than a second threshold value.

8. The processor device for an endoscope according to claim 5,
wherein the power reception information is voltage information showing a direct current voltage corresponding to the load state of the endoscope, and
wherein the power supply control unit controls the frequency of the electric current made to flow to the power transmitting coil such that the electric current moves in a direction approaching a resonant frequency at which the power supply unit and the power receiving unit of the endoscope magnetically resonate with each other, when the voltage information received by the first optical communication unit is lower than a first threshold value, and controls the frequency of the electric current made to flow to the power transmitting coil such that electric current moves in a direction away from the resonant frequency, in the case where the voltage information received by the first optical communication unit is higher than a second threshold value.

9. The processor device for an endoscope according to claim 6,
wherein the power reception information is voltage information showing a direct current voltage corresponding to the load state of the endoscope, and
wherein the power supply control unit controls the frequency of the electric current made to flow to the power transmitting coil such that the electric current moves in a direction approaching a resonant frequency at which the power supply unit and the power receiving unit of the endoscope magnetically resonate with each other, in the case where the voltage information received by the first optical communication unit is lower than a first threshold value, and controls the frequency of the electric current made to flow to the power transmitting coil such that electric current moves in a direction away from the resonant frequency, in the case where the voltage information received by the first optical communication unit is higher than a second threshold value.

10. The processor device for an endoscope according to claim 7,
wherein the first threshold value is a value that is smaller than the second threshold value by a hysteresis width.

11. The processor device for an endoscope according to claim 8,
wherein the first threshold value is a value that is smaller than the second threshold value by a hysteresis width.

12. The processor device for an endoscope according to claim 9,
wherein the first threshold value is a value that is smaller than the second threshold value by a hysteresis width.

13. The processor device for an endoscope according to claim 1, further comprising:
an image signal receiving unit that receives an image signal, which has been captured by the endoscope, in a contactless manner from an image signal transmitting unit provided in the endoscope;
an image processing unit that performs image processing on the image signal received by the image signal receiving unit; and
an output unit that outputs the image signal, which has been subjected to image processing by the image processing unit, to a display.

14. The processor device for an endoscope according to claim 2, further comprising:
an image signal receiving unit that receives an image signal, which has been captured by the endoscope, in a contactless manner from an image signal transmitting unit provided in the endoscope;
an image processing unit that performs image processing on the image signal received by the image signal receiving unit; and
an output unit that outputs the image signal, which has been subjected to image processing by the image processing unit, to a display.

15. The processor device for an endoscope according to claim 1, further comprising:
a light source that supplies light for illumination to a light guide of the endoscope via a mounting unit on which the endoscope is mounted.

16. An endoscope system comprising:
the processor device for an endoscope according to claim 1; and
an endoscope connected to the processor device for an endoscope,
wherein the endoscope includes
a power receiving unit including a power receiving coil that performs power reception in a contactless manner from the processor device for an endoscope, and
a second optical communication unit that starts optical communication with the first optical communication unit provided in the processor device for an endoscope and transmits the power reception information corresponding to an excess or deficiency in the amount of received power of the endoscope through optical communication, if power is supplied via the power receiving unit.

17. The endoscope system according to claim 9,
wherein the processor device for an endoscope includes a control unit that controls an imaging operation of the endoscope, and
wherein the control unit transmits a control signal for controlling the imaging operation of the endoscope from the first optical communication unit to the second optical communication unit.

18. The endoscope system according to claim 16,
wherein the endoscope includes an image signal transmitting unit that transmits an image signal, which has been captured by the endoscope, in a contactless manner to the image signal receiving unit provided in the processor device for an endoscope.

19. A contactless power supply method for an endoscope system including a processor device according to claim 1 for an endoscope having a power supply unit including a power transmitting coil, and a first optical communication unit, and an endoscope having a power receiving unit including a power receiving coil, and a second optical communication unit, the contactless power supply method comprising:
a step of starting power supply from the power supply unit to the power receiving unit of the endoscope if an external trigger receiving unit of the processor device for an endoscope receives an external trigger showing a start instruction for power supply to the endoscope;
a step of causing, if power supply is performed via the power receiving unit, the second optical communication unit to start optical communication with the first optical communication unit provided in the processor device for an endoscope and receive power reception information corresponding to an excess or deficiency in the amount of received power of the endoscope through optical communication; and
a step of causing a power supply control unit provided in the processor device for an endoscope to control the power supply unit on the basis of the power reception information received via the first optical communication unit, and adjust the power to be transmitted from the power supply unit.

20. The contactless power supply method for an endoscope system according to claim 12,
wherein the professor device for an endoscope supplies power to the endoscope in a contactless manner using an electromagnetic resonance method, and
wherein, if the external trigger receiving unit receives the external trigger, the power supply control unit starts power supply from the power supply unit to the power receiving unit of the endoscope, acquires the power reception information from the second optical communication unit via the first optical communication, controls the frequency of an electric current made to flow to the power transmitting coil of the power supply unit on the basis of the acquired power reception information, and adjusts the power to be transmitted from the power supply unit.
